(19) United States
(12) Patent Application Publication
(10) Pub. No.: US 2010/0030335 A1
(43) Pub. Date: Feb. 4, 2010

(54) Compliant Implantable Prosthetic Joint With Preloaded Spring

(75) Inventor: Yves Arramon, Sunnyvale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: SpinalMotion, Inc., Mountain View, CA (US)

(21) Appl. No.: 12/358,726

(22) Filed: Jan. 23, 2009

Related U.S. Application Data
(60) Provisional application No. 61/023,536, filed on Jan. 25, 2008.

(51) Int. Cl. A61F 2/44; A61F 2/28

(52) U.S. Cl. 623/17.13; 623/16.11; 623/17.16

(57) ABSTRACT

An implantable prosthetic joint has a first component for attaching to a first bone and a second component for attaching to a second bone wherein the first and second components are connected in an articulating manner to provide the motion of a prosthetic joint. The joint includes at least one spring to provide compliance to the prosthetic joint. The at least one spring is preloaded in the artificial joint such that the spring is not allowed to move to a completely relaxed position. This preloading of the spring allows the maximum deflection of the spring to be used for shock absorption because the spring does not deform substantially when the implanted prosthetic joint is moved from an at rest position to a loaded or standing position. The prosthetic joints according to the present invention can include artificial hips, knees, shoulders, ankles, and intervertebral discs.

### CROSS-REFERENCES TO RELATED APPLICATIONS

This application claims priority to U.S. Provisional Application No. 61/023,536 filed Jan. 25, 2008, entitled “INTERVERTEBRAL PROSTHETIC DISC WITH SHOCK ABSORBING CORE FORMED WITH DISC SPRINGS;” the full disclosure of which is incorporated herein by reference.

### BACKGROUND OF THE INVENTION

The present invention relates to medical devices and methods. More specifically, the invention relates to implantable prosthetic joints.

The replacement of damaged joints such as knees, hips, shoulders, ankles and intervertebral discs is becoming commonplace to provide patients decreased pain and increased motion after joint deterioration or injury. Many of the artificial joints which are available are made of rigid components, such a metal or polymer ball and socket joints having no compliance or shock absorption. However, in order to accurately mimic natural joint motion, a joint should provide both articulating motion and shock absorption or compliance.

This shock absorption can be provided by either springs or by resilient materials. Since the resilient materials that are available for use in the human body are limited in number and have limited life spans in the fluid environment of the body, metal springs are a better long term solution to providing compliance in an artificial joint.

One example of an artificial joint in which compliance is useful is the intervertebral disc. The known artificial intervertebral discs generally include upper and lower plates or shells which locate against and engage the adjacent vertebral bodies, and a core for providing motion between the plates. The core may be movable or fixed, metallic, ceramic or polymer and generally has at least one convex outer surface which mates with a concave recess on one of the plate in a fixed core device or both of the plates for a movable core device such as described in U.S. Patent Application Publication No. 2006-0025862. However, currently available artificial intervertebral discs do not provide for cushioning or shock absorption which would help absorb forces applied to the prosthesis from the vertebrae to which they are attached. A natural disc is largely fluid which compresses to provide cushioning. It would be desirable to mimic some of this cushioning in an artificial disc. Likewise it would be desirable to mimic the natural cushioning of other natural joints in artificial joint designs.

Therefore, a need exists for improved prosthetic joints. Ideally, such improved joints would provided shock absorption.

### BRIEF SUMMARY OF THE INVENTION

Embodiments of the present invention provide a prosthetic joint with a preloaded metallic spring for shock absorption and methods of providing shock absorption with a prosthetic joint. The prosthetic joint comprises supports that can be positioned against bone and a preloaded spring providing shock absorption between the supports.

In a first aspect an implantable prosthetic joint includes upper and lower supports and a core positioned between the upper and lower supports. The upper and lower supports each include an outer surface which engages a bone and an inner bearing surface. The core is movable with respect to the upper and lower supports and includes upper and lower core members configured to engage the inner bearing surfaces of the upper and lower support plates, and at least one spring in the core between the upper and lower core members to provide compliance to the core. The at least one spring is preloaded in an implantation configuration such that the at least one spring is not allowed to move to a completely relaxed position.

In accordance with another aspect of the invention, an implantable prosthetic joint includes a first component for attaching to a first bone, the first component having a bone engaging surface configured to engage bone and a bearing surface; a second component for attaching to a second bone, the second component having a bone engaging surface configured to engage bone and a bearing surface, and at least one spring positioned in either the first or second component to provide compliance to the prosthetic joint. The bearing surfaces of the first and second components are connected in an articulating manner and the at least one spring is preloaded in an implantation configuration such that the spring is not allowed to move to a completely relaxed position, and the spring does not deform substantially when the implanted prosthetic joint is moved from an at rest position to a loaded position with the patient standing.

In accordance with a further aspect of the invention, a method of assembling a compliant artificial joint includes the steps of: providing upper and lower joint members and an articulating member; positioning at least one spring between the upper and lower joint members in an arrangement which allows the upper and lower joint members to move resiliently toward and away from each other; and locking the upper and lower joint members together in a manner which traps the at least one spring in place between the upper and lower joint members in a preloaded configuration.

### BRIEF DESCRIPTION OF THE DRAWINGS

FIG. 1 is a side cross sectional view of an artificial disc with a shock absorbing core including a plurality of curved spring washers;

FIG. 2 is a perspective view of the shock absorbing core of FIG. 1;

FIG. 3 is a perspective view of a shock absorbing core with flat washers;

FIG. 4 is a cross sectional view the shock absorbing core of FIG. 3 having flat washers;

FIG. 5A is a perspective view a flat washer used in the shock absorbing core of FIG. 3;

FIG. 5B is a cross sectional view the flat washer used in the shock absorbing core of FIG. 3;

FIG. 6 is a perspective view of a shock absorbing core with flat washers as in FIG. 3 rearranged in a parallel and series arrangement;

FIG. 7 is a cross sectional view the shock absorbing core of FIG. 6;

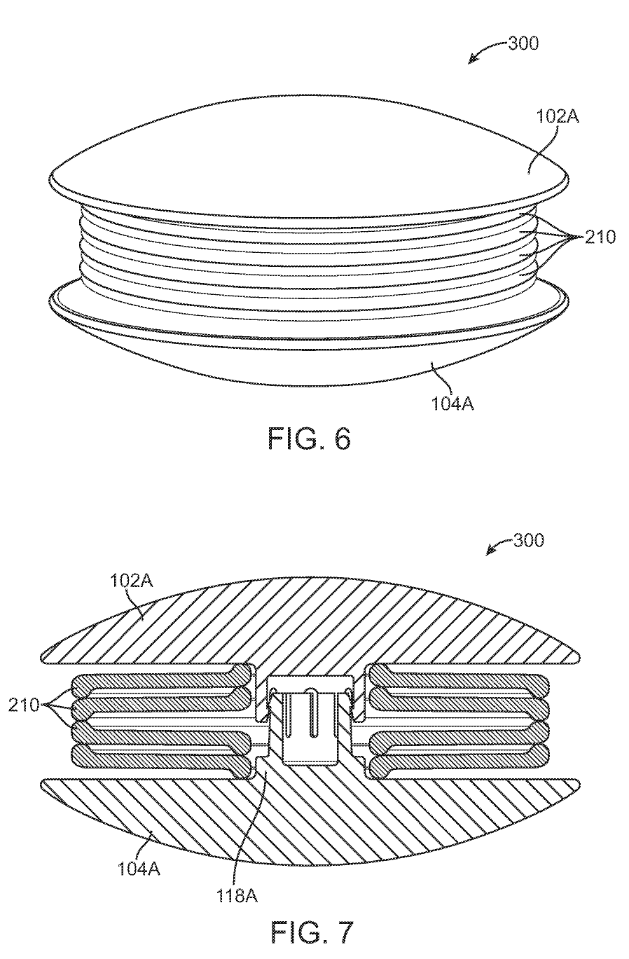

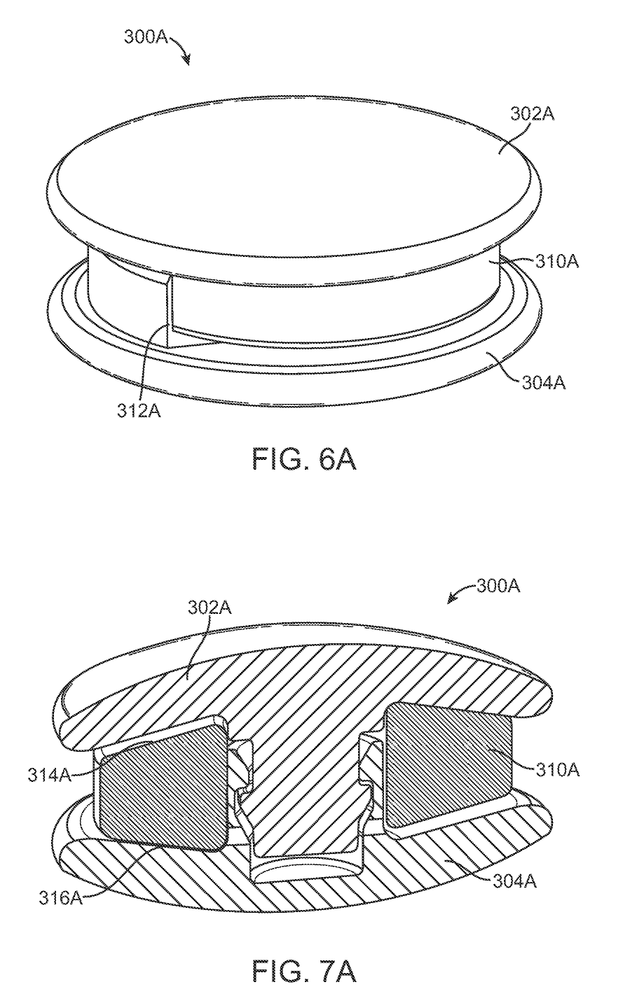

FIG. 6A is a perspective view of a further shock absorbing core with a split ring spring washer;

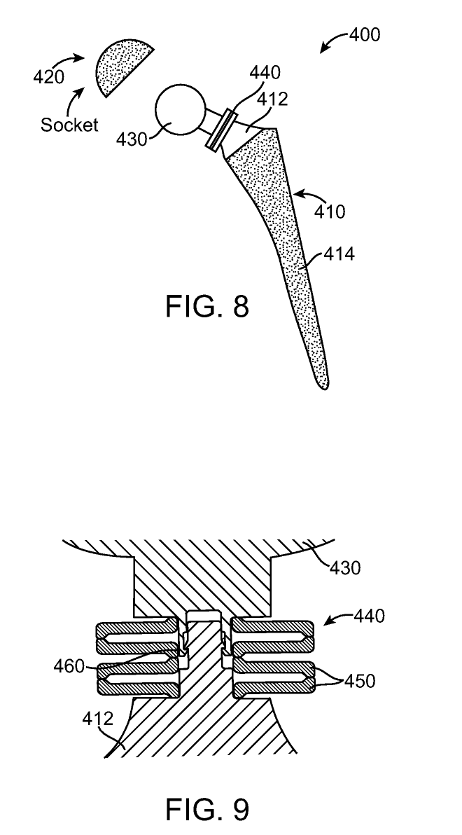

FIG. 7A is a cross sectional view of the core of FIG. 6A;

FIG. 8 is a side view of an artificial hip joint having a shock absorbing shank; and

FIG. 9 is a cross section of the shock absorbing shank of the artificial hip joint of FIG. 8.

### DETAILED DESCRIPTION OF THE INVENTION

Embodiments of the present invention generally provide for implantable prosthetic joints having a first component for attaching to a first bone and a second component for attaching to a second bone wherein the first and second components are connected in an articulating manner to provide the motion of a prosthetic joint. The joint includes at least one spring to provide compliance to the prosthetic joint. The at least one spring is preloaded in the artificial joint such that the spring is not allowed to move to a completely relaxed position. This preloading of the spring allows the maximum deflection of the spring to be used for shock absorption because the spring does not deform substantially when the implanted prosthetic joint is moved from an at rest position to a loaded or standing position. The prosthetic joints according to the present invention can include artificial hips, knees, shoulders, ankles, and intervertebral discs.

The term “preloaded” as used herein means the spring is loaded or deformed from its initial relaxed configuration by loading the spring with a preload force.

The non-loaded spring begins deforming at a small load, for example the non-loaded spring begins deforming when the patient moves from rest to standing. The non-loaded spring may then bottom out when additional forces of impact are applied beyond standing. For a prosthetic joint with a non-loaded spring, the total amount of spring deformation available in the joint for absorbing impacts is used for both standing and for impacts upon standing. In contrast, a prosthetic joint with a preloaded spring requires a larger load for initial deflection and allows the majority of the deflection to be available to absorb impacts at higher forces than those occurring during standing. This provides the added benefit that the artificial joint kinematics remain unchanged from kinematics of a joint without a spring during ordinary behavior, i.e. standing, sitting, and possibly walking. The compliance or deflection of the spring elements is needed primarily for impacts beyond this ordinary behavior.

In one example, embodiments of the present invention generally provide for an artificial intervertebral disc having upper and lower plates disposed about a shock absorbing mobile core. The shock absorbing core includes one or more spring washers or disc springs between upper and lower surfaces of the core to allow the upper and lower surfaces to move resiliently toward and away from each other. This allows the core to absorb forces applied to it by the vertebrae.

Intervertebral discs must fit into the space between adjacent vertebrae. This intervertebral space is on the order of 1-2 cm in the lumbar region and half of that in the cervical region. This restricted space leaves very little room to accommodate a spring element to provide desired compliance to an artificial disc design. Spring washers or disc springs made of metal are particularly suited for this application to provide a compliant element that can accommodate a relatively large load within a very small height. However, other non-metallic spring elements may also be used including polymer springs and elastomeric elements. Helical springs can also be used in some applications, however the thinner configuration of springs washers and disc springs can accommodate greater force in a smaller height.

The shock absorbing cores described herein can be used with many artificial disc designs and with different approaches to the intervertebral disc space including anterior, lateral, posterior and posterior lateral approaches. Although various embodiments of such an artificial disc are shown in the figures and described further below, the general principles of these embodiments, namely providing a force absorbing design with a preloaded spring, may be applied to any of a number of other disc prostheses, as well as the other types of prosthetic joints mentioned herein.

Spring washers or disc springs as used in the shock absorbing joints of the present invention are particularly well suited for carrying large loads in very small spaces and for providing small deflections and extremely long fatigue life. Spring washers come in a variety of configurations and can be stacked in different manners to tailor the loads and deflections to a particular application. Although spring washers are generally disc shaped members with a central hole, as will be seen below, the hole may be omitted in some cases to form disc springs. In addition, the washers or discs may be slotted, split, or contoured in a variety of ways.

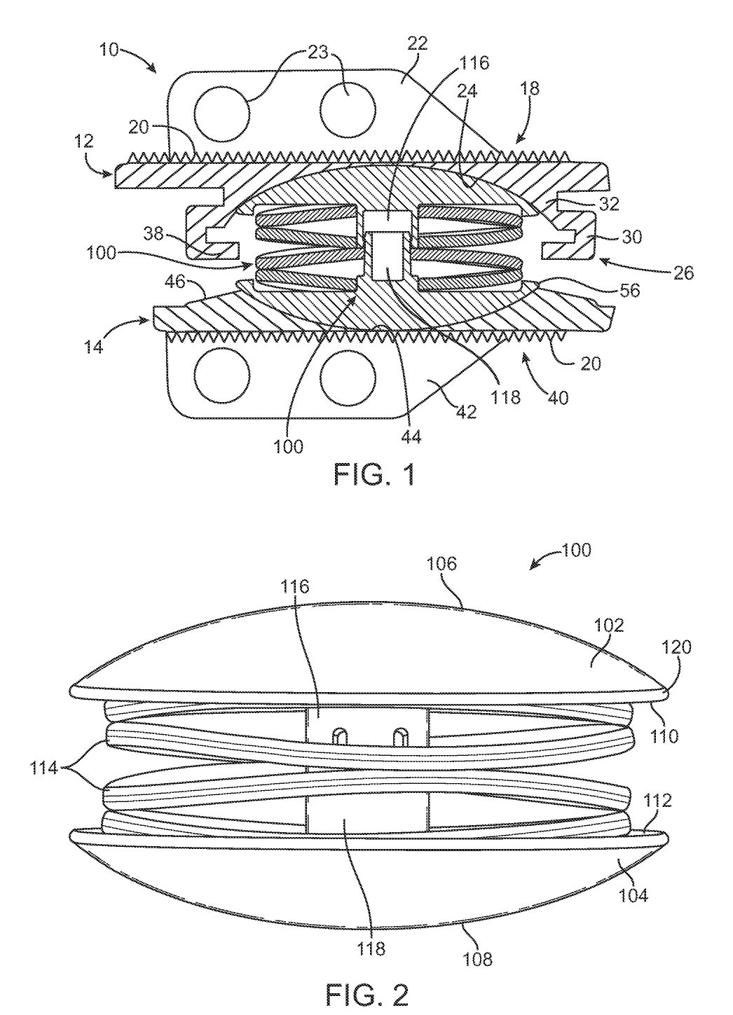

FIG. 1 shows an artificial disc 10 having a shock absorbing core 100, according to one embodiment of the present invention. The disc 10 for intervertebral insertion between two adjacent spinal vertebrae (not shown) includes an upper plate 12, a lower plate 14 and the movable shock absorbing core 100 located between the plates. The upper plate 12 includes an outer bone engaging surface 18 and an inner bearing surface 24 and may be constructed from any suitable metal, alloy or combination of metals or alloys, such as but not limited to cobalt chrome molybdenum alloys, titanium (such as grade 5 titanium), stainless steel and/or the like. In one embodiment, typically used in the lumbar spine, the upper plate 12 is constructed of cobalt chrome molybdenum, and the outer surface 18 is treated with aluminum oxide blasting followed by a titanium plasma spray. In another embodiment, typically used in the cervical spine, the upper plate 12 is constructed of titanium, the inner surface 24 is coated with titanium nitride, and the outer surface 18 is treated with aluminum oxide blasting. An alternative cervical spine embodiment includes no coating on the inner surface 24. In other cervical and lumbar disc embodiments, any other suitable metals or combinations of metals may be used. In some embodiments, it may be useful to couple two materials together to form the inner surface 24 and the outer surface 18. For example, the upper plate 12 may be made of an MRI-compatible material, such as titanium, but may include a harder material, such as cobalt chrome molybdenum, for the inner surface 24. In another embodiment, upper plate 12 may comprise a metal, and inner surface 24 may comprise a ceramic material. All combinations of materials including metals and other rigid materials, such a polyetheretherketone (PEEK) are contemplated within the scope of the present invention. Any suitable technique may be used to couple materials together, such as snap fitting, slip fitting, lamination, interference fitting, use of adhesives, welding and/or the like. Any other suitable combination of materials and coatings may be employed in various embodiments of the invention.

In some embodiments, the outer surface 18 is planar. Oftentimes, the outer surface 18 will include one or more surface features and/or materials to enhance attachment of the prosthesis 10 to vertebral bone including serrations, fins, coatings, teeth, or threaded fasteners. For example, the outer surface 18 may be machined to have serrations 20 or other surface features for promoting adhesion of the upper plate 12 to a vertebra. In the embodiment shown, the serrations 20 extend in mutually orthogonal directions, but other geometries would also be useful. Additionally, the outer surface 18 may be provided with a rough microfinish formed by blasting with aluminum oxide microparticles or the like. In some embodiments, the outer surface may also be titanium plasma sprayed to further enhance attachment of the outer surface 18 to vertebral bone.

The outer surface 18 may also carry one or more upstanding, vertical fins 22 extending in an anterior-posterior direction. In one embodiment, the fin 22 is pierced by transverse holes 23 for bone ingrowth. In alternative embodiments, the fin 22 may be rotated away from the anterior-posterior axis, such as in a lateral-lateral orientation, a posterolateral-anterolateral orientation, or the like depending on the direction of insertion of the disc. In some embodiments, the fin 22 may extend from the surface 18 at an angle other than 90°. Furthermore, multiple fins 22 may be attached to the surface 18 and/or the fin 22 may have any other suitable configuration, in various embodiments. In some embodiments, such as discs 10 for cervical insertion, the fins 22, 42 may be omitted altogether.

The inner, spherically curved concave surface 24 provides a bearing surface for the shock absorbing core 100. At the outer edge of the curved surface 24, the upper plate 12 carries a peripheral restraining structure comprising an integral ring structure 26 including an inwardly directed rib or flange 38. The flange 38 forms part of a U-shaped member 30 joined to the major part of the plate by an annular web 32.

The lower plate 14 is similar to the upper plate 12 except for the absence of the peripheral restraining structure 26. Thus, the lower plate 14 has an outer surface 40 which is planar, serrated and microfinished like the outer surface 18 of the upper plate 12. The lower plate 14 optionally carries one or more fins 42 similar to the fin 22 of the upper plate. The inner surface 44 of the lower plate 14 is concavely, spherically curved with a radius of curvature matching that of the shock absorbing core 100 to provide a bearing surface for the core. Once again, the inner surface 44 may be provided with a titanium nitride or other finish.

At the outer edge of the inner curved surface 44, the lower plate 14 is provided with an inclined ledge formation 46 which contacts the flange 38 of the upper plate to limit the range of motion of the plates. Alternatively, the lower plate 14 may include a peripheral restraining structure analogous to the peripheral restraining structure 26 on the upper plate 12.

The shock absorbing core 100 shown in FIGS. 1 and 2 and described herein includes upper and lower core members 102, 104 which are symmetrical about a central, equatorial plane. In other embodiments, the shock absorbing core 100 may be asymmetrical. The upper and lower core members 102, 104 include convexly curved outer surfaces 106, 108 configured to cooperate with the bearing surfaces 24, 44 of the upper and lower plates 12, 14. Opposite the convex outer surfaces 106, 108 of the core members are inner surfaces 110, 112 arranged to form contact surfaces for spring washers. Between the upper and lower core members 102, 104 are one or more spring washers 114, which in the embodiment of FIGS. 1 and 2 are four curved spring washers. The inner surfaces 110, 112 can be flat or contoured depending on the shape of the spring washers and the space needed for the one or more washers. For example, where more space is needed for the spring washers, the inner surfaces 110, 112 may be recessed within the upper and/or lower core members wherein form a chamber to accommodate at least a portion of the one or more washers 114.

When the plates 12, 14 and shock absorbing core 100 are assembled and in the orientation seen in FIG. 1, a rim or lip 120 of the upper core member 102 fits above the flange 38 on the upper plate 12 so that as the core moves within the disc 10 it is retained by the flange 38. The flange 38 prevents separation of the core 100 from the plates. In other words, the cooperation of the retaining formations of the upper plate flange 38 and the lip 120 ensures that the shock absorbing core 100 is held captive between the plates 10, 14 at all times during flexure of the disc 10.

The outer diameter of the lips 120 on the core are very slightly smaller than the diameter defined by the inner edge of the flange 38 to allow the core to be placed into the opening in the top plate 12. In another embodiment, the shock absorbing core 100 is movably fitted into the upper plate 12 via an interference fit. To form such an interference fit with a metal core 100 and metal plate 12, any suitable techniques may be used. For example, the plate 12 may be heated so that it expands, and the core 100 may be dropped into the plate 12 in the expanded state. When the plate 12 cools and contracts the interference fit is created. In another embodiment, the upper plate 12 may be formed around the shock absorbing core 100. Alternatively, the shock absorbing core 100 and upper plate 12 may include complementary threads, which allow the shock absorbing core to be screwed into the upper plate 12, where it can then freely move.

In an alternative embodiment, the continuous annular flange 38 may be replaced by a retaining formation comprising a number of flange segments which are spaced apart circumferentially. In yet another embodiment, the retaining formation(s) can be carried by the lower plate 14 instead of the upper plate 12, i.e. the plates are reversed. In some embodiments, the upper (or lower) plate is formed with an inwardly facing groove, or circumferentially spaced groove segments, at the edge of its inner, curved surface, and the outer periphery of the core 100 is formed with an outwardly facing flange or with circumferentially spaced flange segments.

In use the disc 10 is surgically implanted between adjacent spinal vertebrae in place of a damaged disc which has been removed by a known discectomy procedure. The adjacent vertebrae are forcibly separated from one another to provide the necessary space for insertion. The disc 10 is typically, though not necessarily, advanced toward the disc space from an anterolateral or anterior approach and is inserted in a posterior direction—i.e., from anterior to posterior. The disc 10 is inserted into place between the vertebrae with the fins 22, 42 of the top and bottom plates 12, 14 entering slots cut in the opposing vertebral surfaces to receive them. During and/or after insertion, the vertebrae, facets, adjacent ligaments and soft tissues are allowed to move together to hold the disc in place. The serrated and microfinished surfaces 18, 40 of the plates 12, 14 locate against the opposing vertebrae. The serrations 20 and fins 22, 42 provide initial stability and fixation for the disc 10. With passage of time, enhanced by the titanium or other surface coating, firm connection between the plates and the vertebrae will be achieved as bone tissue grows over the serrated surface. Bone tissue growth will also take place about the fins 22, 40 and through the transverse holes 23 therein, further enhancing the connection which is achieved.

In the assembled disc 10, the complementary and cooperating spherical surfaces of the plates 12, 14 and shock absorbing core 100 allow the plates to slide or articulate over the core through a fairly large range of angles and in all directions or degrees of freedom, including rotation about the central axis. FIG. 1 shows the disc 10 with the plates 12 and 14 and shock absorbing core 100 aligned vertically with one another.

Preloading of the springs in the present invention can be performed in the embodiment of FIG. 1 by placing the springs between the upper and lower core members 102, 104 in a configuration in which the cores are unloaded and loading the cores while snapping the retention features together. Preloading provides, among other advantages, the advantage that the entire deflection available in the core is available for shock absorption of shocks due to impacts at the standing position. Preloading can substantially eliminate the compression of the core that would otherwise occur during a change from lying to standing position. Preloading can also reduce or eliminate the stretching of the ligaments around the joint by the extension of the spring when the joint is unloaded. For example, without preloading the joint may be stretched by the spring when the patient lies down and this stretching could over time loosen the joint.

### Curved Spring Washers

Referring now to FIGS. 1 and 2, a shock absorbing core 100 is shown in which the core includes a plurality of curved spring washers 114. The curved spring washers 114 can be as simple as flat disc shaped washers which are bent in a single direction. The elasticity of the curved spring washers 114 is a result of the resistance of the washers to flattening. The embodiment of FIGS. 1 and 2 shows four curved washers 114 arranged in series (with alternating directions of curvature). The washers 114 are preferably oriented at a fixed orientation so that the peaks of the adjacent washers remain in contact with each other to achieve consistent deflections. When the washers 114 are compressed during loading, the diameter of the washers expand.

As shown in FIG. 1, the upper and lower disc members 102, 104 include telescoping central posts 116, 118 which snap together with a retention feature which will be described in further detail below. The four curved spring washers 114 are placed over the central telescoping posts 116, 118 before the retention features are snapped together. A total deflection of the compliant core 100 is determined by the curvature of the curved washers 114. The inner surfaces 110, 112 of the upper and lower disc members 102, 104 are preferably formed of a hard material to form a bearing surface which is resistant to wear by the washers which may tend to dig into the bearing surface due to their shape. Although the curved washers 114 are illustrated as circular washers, they can be formed in other shapes, such as rectangular washers or circular washers with truncated or flattened ends.

### Flat Spring Washers

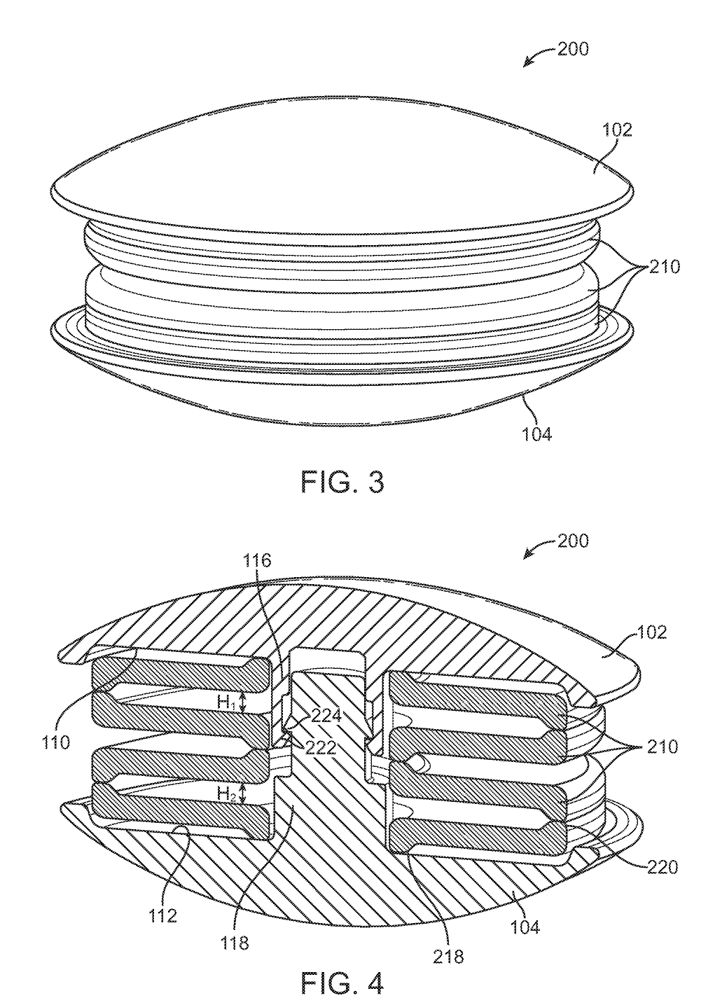

FIGS. 3-7 illustrate variations of a shock absorbing core 200 with flat spring washers 210 which are deflected out of their flat configuration into domed or conical shapes by application of a load to the core. Flat spring washers 210 can be flat or essentially flat with one or more ribs, rings, lips or other features for transferring forces to and from the flat washers. The resilience of the flat spring washers 210 is a result of resistance of the spring to moving out of the flat configuration. The flat spring washers 210 may include curves and bends or slots which will change the stiffness of the springs.

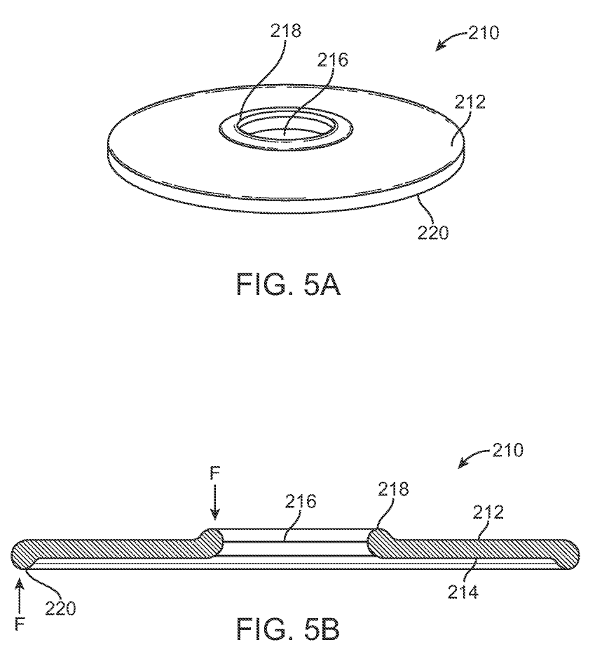

FIGS. 5A and 5B illustrate a single flat washer 210 having a substantially flat top surface 212 and a substantially flat bottom surface 214. The top and bottom surfaces 212, 214 can have a somewhat contoured surface to distribute strain within the washer 210. The washer 210 has a central hole 216 and an inner lip 218 surrounding the hole. The inner lip 218 may be spaced from the hole or the hole may be omitted all together in some designs. An outer periphery of the washer 210 has an outer lip 220 which extends from the flat portion of the washer 210 in an axial direction opposite of the inner lip 218. Application of a force to the spring washer 210 at the lip portions 218, 220 in the directions of the arrows F shown in FIG. 5B causes the spring washer to deform out of its original plane. Removal of the force F allows the spring washer to return to its preloaded shape.

The flat spring washers 210 are shown in FIGS. 3-8 have rims or lips 218, 220 for transferring forces to the washers and for spacing the washers from adjacent structures in the core to allow the washers space to deform. Although the ribs 218, 220 for transferring forces to the washers 210 are shown on the washers themselves, these ribs can alternatively or additionally be formed on the inner contact surfaces 110, 112 of the upper and lower core members 102, 104. The rims 218, 220 may be replaced with other force transferring structures on the washers or on adjacent structures. For example, in a core with a single flat spring washer, the washer may be flat and the upper and lower core members 102, 104 may be provided with projecting rims one near the inner edge of the washer and one near the outer edge of the washer.

The compliant core 200 shown in FIG. 4 has a locking retention feature in the form of the telescoping central posts 116, 118 with mating snap lock projections 222, 224. When the telescoping posts 116, 118 are in the locked position, the washers 210 are arranged in preloaded configuration in which the washers are tightly packed and deformed with a force that approximates the weight of a standing patient. A preloaded configuration provides the advantage of a compliant disc which can be designed to have the same height when the patient is at rest and at stance. The preloading minimizes or eliminates compression of the core while the patient is standing up and maintains all of the potential core deflection for the impact loads after standing that the disc is designed to absorb.

The amount of maximum total deflection provided by a core such as the core 200 in FIG. 4 is measured as a total of the heights H₁ and H₂ between the washers. When the washers 210 are placed in series as in FIG. 4, the four washers provide four times the deflection of a single washer for the same stress. Washers may also be arranged in parallel as will be shown in FIGS. 6 and 7 to increase the load bearing capacity.

FIGS. 6 and 7 illustrate a shock absorbing core 300 having upper and lower core members 102A and 104A and four flat spring washers 210. The upper and lower core members 102A, 104A of the core 300 have flat inner surfaces and modified central posts from the core 200 of FIG. 4, however, the core works in a similar manner. The flat washers 210 as seen in FIG. 7 are arranged with the top two washers in a parallel arrangement and the bottom two washers in a parallel arrangement to increase the load bearing capacity of the core. The upper and lower pairs of washers 210 are arranged in series.

The core designs described herein can all be modified to provide more stability in shear by modification of the central posts or other retention feature. Stiffness can be increased or decreased as necessary by increasing or decreasing the thickness of the spring washers or disc springs. Compliance can be increased or decreased by modifying the spacing between the spring washers or disc springs.

### Split Spring Coil Washer

Another version of a core with a split spring washer is shown in FIGS. 6A and 7A. A shock absorbing core 300A includes upper and lower core members 302A, 304A and a single split spring washer 310A. This single split spring washer embodiment is particularly useful in smaller applications, such as the cervical application where the total height of the core may be on the order of 5 mm. As shown in FIG. 6A, the split washer 310A is bent in the manner of a common lock washer to form what is essentially just less than one turn of a coil spring. This results in an offset of the ends of the split washer at the location of the split 312A in the washer. In operation, as the upper and lower core members 302A, 304A move toward each other while the core 300A is compressed, the split spring washer 310A deforms from the offset or coil configuration to a flat configuration. Upper and lower surfaces 314A and 316A of the washer 310A and corresponding surfaces of the upper and lower core members 302A, 304A are tapered to assist in retaining the washer within the core 300A. However, other shapes of these surfaces may also be employed.

The tapered shape of the split spring washer 310A in cross section and corresponding shape of the upper and lower core members provides a safety feature by trapping the spring in case the spring becomes fractured.

Additional shapes of spring washers and spring discs are shown in U.S. Provisional Patent Application Ser. No. 61/023,480, filed Jan. 25, 2008, entitled “INTERVERTEBRAL PROSTHETIC DISC WITH SHOCK ABSORBING CORE FORMED WITH DISC SPRINGS” and U.S. Provisional Patent Application No. 61/049,259 filed Apr. 30, 2008, entitled “INTERVERTEBRAL PROSTHETIC DISC WITH SHOCK ABSORBING CORE FORMED WITH DISC SPRINGS” the full disclosures of which are incorporated herein by reference. These same springs and others in a preloaded configuration can be used in other types of prosthetic joints including hips, knees, shoulders, ankles, and the like.

FIGS. 8 and 9 illustrate one example of a prosthetic hip joint with a preloaded spring arrangement which provide the hip joint with compliance. The ball and socket type hip joint 400 of FIG. 8 includes a femoral implant 410 and a socket implant 420 which are implanted into the opposing bones of the hip and mate together to provide a rotating prosthetic joint. The femoral implant 410 includes a spring element 440 positioned in a shank 412 of the implant between an implantable spike 414 and a ball 430. As shown in the enlarged illustration of FIG. 9, the spring element 440 includes a plurality of stacked flat spring washers 450 similar to those shown in FIG. 3. The shank 412 includes a snap lock retention feature 460 which locks together to hold the springs 450 in the preloaded configuration.

In ball and socket type prosthetic joints as shown in FIGS. 8 and 9 the preloaded spring element can be incorporated into the ball or the socket portion of the artificial joint and can be in the form of any of the different spring elements described herein.

Preferably the preloaded springs in the artificial joints of the present invention are made of metal such as titanium, cobalt chromium alloy, stainless steel, NiTi or a combination thereof. These materials provide a high hardness surface for the upper and lower surfaces of the cores which improve performance and prevent particulate generation. These materials also can be designed to provide spring washers which are deformable in the elastic region of the stress/strain curve and will not plastically deform during compression.

The materials, shape, size, number, arrangement (series or parallel stacking) and other features of the preloaded springs can be varied to provide tailored compliance properties for different kinds applications. Spring washers or spring disc may be provided in a plurality of different configurations including curved spring washers or discs, flat spring washers or discs, wave springs, cone shaped washers, slotted washers, and the like. The preloaded springs can also be modified to provide tailored behaviors, such as non linear spring behaviors providing progressively stiffer behavior upon larger compression.

The prosthetic joints preferably have a predesigned hard stop so that the maximum amount of deflection of the springs is limited to a predetermined amount according to the application.

Preloading of the springs in the present invention can be performed by placing the springs between two elements of the prosthetic joint and snapping a retention feature together. The retention features shown are merely one example of the type of retention feature that can be used. Preloading provides, among other advantages, the advantage that the entire deflection available in the spring is available for shock absorption at the standing position. Preloading can eliminates the compression of the core that would otherwise occur during a change from lying to standing position.

In one embodiment of the present invention, for a cervical prosthetic intervertebral disc, the maximum deformation of the shock absorbing disc is about 0.1 to about 1.0 mm, and is preferably about 0.2 to about 0.8 mm. The amount of force to deflect the cervical disc from the initial preloaded configuration is about 10 N to about 50 N and the amount of force to complete deflection or bottom out is about 100 N to about 1000 N.

For a lumbar prosthetic intervertebral disc, the maximum deformation of the shock absorbing disc is about 0.2 to about 2.0 mm, and is preferably about 0.4 to about 1.5 mm. The amount of force to deflect the lumbar disc from the initial preloaded configuration is about 200 N to about 500 N and the amount of force to complete deflection or bottom out is about 1000 N to about 3000 N.

For a prosthetic hip joint, the maximum deformation of the joint is about 0.2 to about 10.0 mm, and is preferably about 1.0 to about 5 mm. The amount of force to deflect the prosthetic hip joint from the initial preloaded configuration is about 500 N to about 1000 N and the amount of force to complete deflection is about 3000 N to about 5000 N.

While the exemplary embodiments have been described in some detail, by way of example and for clarity of understanding, those of skill in the art will recognize that a variety of modifications, adaptations, and changes may be employed. Hence, the scope of the present invention should be limited solely by the appended claims.

What is claimed is:

1. An implantable prosthetic joint comprising:
upper and lower supports, each support comprising,
an outer surface which engages a bone, and
an inner bearing surface;
a core positioned between the upper and lower supports and movable with respect to the upper and lower supports, the core comprising,
upper and lower core members configured to engage the inner bearing surfaces of the upper and lower support plates, and
at least one spring in the core between the upper and lower core members to provide compliance to the core, and
wherein the at least one spring is preloaded in an implantation configuration such that the at least one spring is not allowed to move to a completely relaxed position.

2. The joint of claim 1, wherein the spring is preloaded by locking the upper and lower core members together.

3. The joint of claim 1, wherein the joint is an artificial intervertebral disc.

4. The joint of claim 3, wherein the at least one spring is preloaded in an implantation configuration such that the spring does not deform substantially when the implanted prosthetic joint is moved from an at rest position to a loaded position with the patient standing.

5. The joint of claim 4, wherein the at least one spring does not deform until the load on the disc is about 10 N.

6. The joint of claim 1, wherein the at least one spring does not deform until the load on the joint is about 10 N.

7. The joint of claim 1, wherein the at least one spring is a disc spring.

8. The joint of claim 1, wherein the at least one spring is a spring washer.

9. The joint of claim 1, wherein the preloaded spring is configured to maintain substantially the same initial position whether the patient is laying, sitting, or standing.

10. An implantable prosthetic joint comprising:
a first component for attaching to a first bone, the first component having a bone engaging surface configured to engage bone and a bearing surface;
a second component for attaching to a second bone, the second component having a bone engaging surface configured to engage bone and a bearing surface, wherein the bearing surfaces of the first and second components are connected in an articulating manner;
at least one spring positioned in either the first or second component to provide compliance to the prosthetic joint; and
wherein the at least one spring is preloaded in an implantation configuration such that the spring is not allowed to move to a completely relaxed position, and the spring does not deform substantially when the implanted prosthetic joint is moved from an at rest position to a loaded position with the patient standing.

11. The joint of claim 10, wherein the spring is preloaded by snapping two parts of the prosthetic joint together in a snap lock configuration.

12. The joint of claim 10, wherein the joint is an artificial intervertebral disc.

13. The joint of claim 10, wherein the at least one spring is a disc spring.

14. The joint of claim 10, wherein the at least one spring is a spring washer.

15. The joint of claim 10, wherein the preloaded spring is configured to maintain substantially the same initial position whether the patient is laying, sitting, or standing.

16. The joint of claim 10, wherein the spring has a maximum deflection of about 0.1 to about 3 mm from an initial preloaded position to a maximum deflected position.

17. The joint of claim 10, wherein the joint is one of a hip, knee, shoulder, and ankle.

18. The joint of claim 10, wherein the second component includes a bone engaging component and a mobile core component, and wherein the mobile core component includes the at least one preloaded spring.

19. A method of assembling a compliant artificial joint, the method comprising:
providing upper and lower joint members and an articulating member;
positioning at least one spring between the upper and lower joint members in an arrangement which allows the upper and lower joint members to move resiliently toward and away from each other; and
locking the upper and lower joint members together in a manner which traps the at least one spring in place between the upper and lower joint members in a preloaded configuration.

20. The method of claim 19, wherein the spring is preloaded such that the spring is not allowed to move to a completely relaxed position and the spring does not deform substantially when the implanted prosthetic joint is moved from an at rest position to a loaded position with the patient standing.

21. The method of claim 20, wherein the spring is preloaded by snapping two parts of the prosthetic joint together in a snap lock configuration.

22. The method of claim 20, wherein the joint is an artificial intervertebral disc.

23. The method of claim 20, wherein the at least one spring is a disc spring.

24. The method of claim 20, wherein the joint is one of a hip, knee, shoulder, and ankle.
